(19) United States
(12) Patent Application Publication
(10) Pub. No.: US 2002/0103169 A1
(43) Pub. Date: Aug. 1, 2002

(54) Chemical derivatives and use thereof as antitelomerase agents

(76) Inventors: Patrick Mailliet, Fontenay Sous Bois (FR); Jean-Francois Riou, Paris (FR); Jean-Louis Mergny, Villejuif (FR); Abdelazize Laoui, Bridgewater, NJ (US)

Correspondence Address: Finnegan, Henderson, Farabow,, Garrett & Dunner, L.L.P., 1300 I Street, N.W., Washington, DC, 20005-3315, US

(21) Appl. No.: 09/923,541

(22) Filed: Aug. 8, 2001

(30) Foreign Application Priority Data
Aug. 8, 2000 (FR) 00 10422

(51) Int. Cl. A61K 31/655; A61K 31/473; C12Q 1/68; C07H 21/04; C09B 29/42; C09B 39/00

(52) U.S. Cl. 514/150; 514/298; 435/6; 534/551; 534/727; 534/800; 536/25.32; 546/108

(57) ABSTRACT

Novel anticancer agents of the phenanthridine class having a specific mechanism of action and cancer therapy methods are described, including the novel chemical compounds and their therapeutic use thereof in humans.

The present invention relates to cancer therapy and novel anticancer agents having a specific mechanism of action. The present invention further relates to novel chemical compounds and the therapeutic use thereof in humans.

In one embodiment, the present invention relates to the use of novel chemical non-nucleotide compounds that interact with specific structures of deoxyribonucleic acid (DNA). These novel compounds consist of phenanthridine derivatives, useful in treating cancers. These novel compounds act as telomerase-inhibiting agents by stabilizing DNA in the G-quadruplex structure (guanine tetrads). The therapeutic application of the inhibition of telomerase via the stabilization of these G-quadruplexes is the arrest of cellular mitosis and the death of rapidly dividing cells such as cancer cells, thereby inhibiting tumor growth and, possibly, the induction of cancer cell senescence.

The compounds of the present invention have the advantage, from the therapeutic point of view, of blocking telomerase. From the biological point of view, telomerase allows the addition of repeat DNA sequences of the TTAGGG type, termed “telomeric sequences”, at the end of the telomere during cell division.

Through this action, telomerase renders the cell immortal. Specifically, in the absence of this enzymatic activity, the cell loses 100 to 150 bases at each division, which rapidly make the cell senescent. When rapidly dividing cancer cells appear, it has become apparent that these cells have telomeres that are maintained at a stable length during cell division. In these cancer cells, it has become apparent that telomerase is highly active and that it allows the addition of repeat motifs of telomeric sequences to the end of the telomere, thereby allowing the length of the telomere to be conserved. It has been apparent for some time that more than 85% of cancer cells test positive in assays for the presence of telomerase, whereas somatic cells do not show this characteristic.

Thus, telomerase is a very coveted target for treating cancer cells. The first obvious approach for blocking telomerase has been the use of nucleotide structures (Chen et al., Proc. Nati. Acad. Sci. 93(7):2635-2639). Among the non-nucleotide compounds that have been used previously, mention may be made of diaminoanthraquinones (Sun et al., J. Med. Chem. 40(14):2113-2116) or diethyloxadicarbocyanins (Wheelhouse R. T. et al., J. Am. Chem. Soc. 120:3261-3262 (1998)).

International Patent Application No. WO 99/40087 describes the use of compounds that interact with G-quadruplex structures, including perylene compounds and carbocyanins containing at least seven rings-two of which are heterocycles.

It has become apparent, most surprisingly, that simple structures make it possible to obtain at least an equivalent result, with structures that are much less complicated from a chemical point of view. The compounds of the present invention that satisfy this objective, i.e., compounds that bind the G-quadruplex structure and, by virtue of this, have telomerase-inhibiting activity, have the following general formula (I):

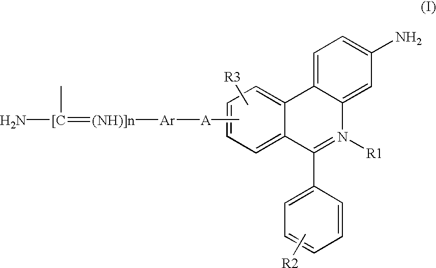

in which

A represents a basic nitrogenous linkage chosen from secondary or tertiary amino, azo, diazo, diazoamino, and hydrazo groups;

Ar represents at least one mono- or polycyclic, carbocyclic or heterocyclic aromatic group containing 4 to 10 carbon atoms, with said aromatic group being optionally substituted with at least one C1-C4 alkyl group;

n represents 0 or 1;

R1 represents a C1-C4 alkyl group;

R2 represents at least one substituent chosen from hydrogen, C1-C4 alkyl, C1-C4 alkoxy, C1-C4 alkylthio, and an amino group; and

R3 represents at least one substituent chosen from hydrogen, C1-C4 alkyl, C1-C4 alkoxy, C1-C4 alkylthio, and an amino group.

Alkyl, alkoxy, and alkylthio represent groups that may be linear or branched.

For the purposes of the formula above, examples of aromatic heterocycles include nitrogenous heterocycles comprising at least one nitrogen atom, such as, for example, pyridine, pyrimidine, or pyrazine groups. Compounds of the formula (I) containing at least 7 nitrogen atoms in their general formula are also within the scope of the invention.

Examples of R1 include methyl or ethyl groups.

Representative compounds offormula (I) include:

isomethamidium, or 7-(3-amidinophenyl)diazoamino-2-amino-10-ethyl-9-phenylphenanthridinium chloride hydrochloride (Example 1),

8-(3-amidinophenyl)diazo-2,7-diamino-10-ethyl-9-phenylphenanthridinium bromide hydrobromide (Example 2),

7-amino-2-(2-amino-1,6-dimethylpyrimidinio-4-yl)-(4-aminophenyl)-10-ethylphenanthridinium bromide (Example 3).

In another embodiment, the present invention also relates to the novel compounds of the following formula:

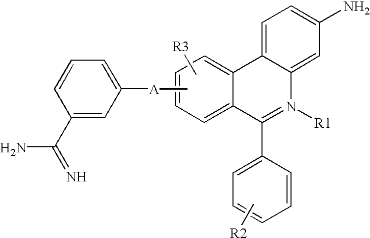

in which

R1 represents a C1-C2 alkyl group,

R2 represents hydrogen or at least one amino group,

R3 represents hydrogen or at least one amino group,

A represents an azo, diazo, diazoamino, hydrazo or amino group.

The process for preparing the compounds of formula (I) above in which A represents a hydrazo group is, for example, described by S. S. Berg in Nature 188:1106-1107 (1960) for the synthesis of isomethamidium (Example 1), which process is incorporated herein by reference.

The novel compounds are prepared by coupling a 3-benzamidodiazonium salt with a 7-aminophenanthridinium salt, according to the following reaction sequence.

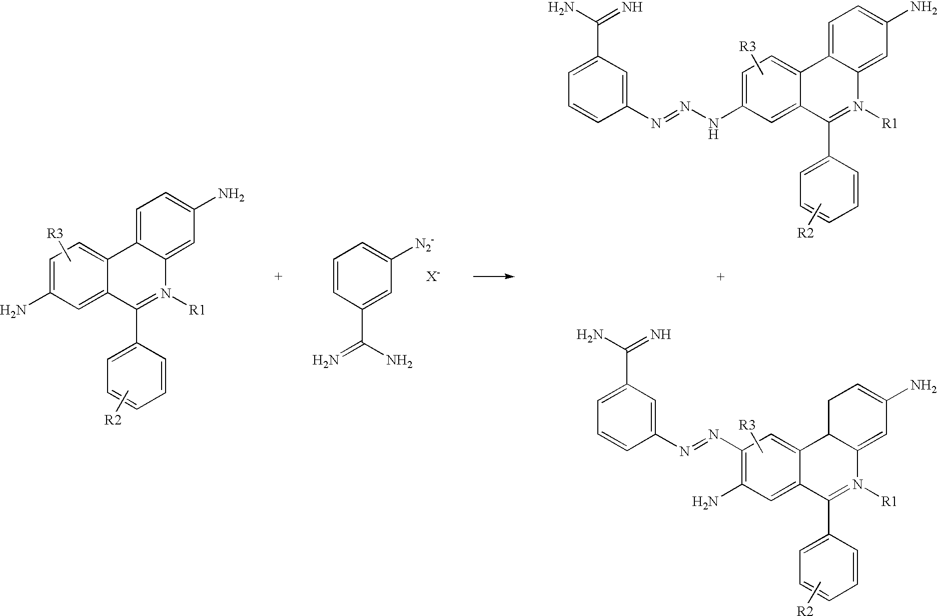

Depending on the substituents present on the 7-aminophenanthridinium salt, the coupling with the 3-benzamidodiazonium salt can also take place in the ortho (or para) position of the 7-amino function, thus producing the compounds of general formula (I) above in which A represents an azo group. For example, the compound of Example 2 can be obtained using such an ortho-azo-coupling of an amine function according to the scheme above.

The products of general formula (I) in which A represents a secondary or tertiary amino group are obtained by carrying out the coupling of an aromatic amine with 2-amino-4-chloro-6-methylpyrimidine, followed by N-methylation of the nitrogen at position 1 of the pyrimidine, proceeding as, for example, under the conditions described by Bukrinsky et al. in International Patent Application No. WO 96/20932. Thus, more specifically, the product of Example 3 is obtained as a by-product of the synthesis of prothidium, from which it is separated by crystallization proceeding as described in GB Patent No. 816236. See the following

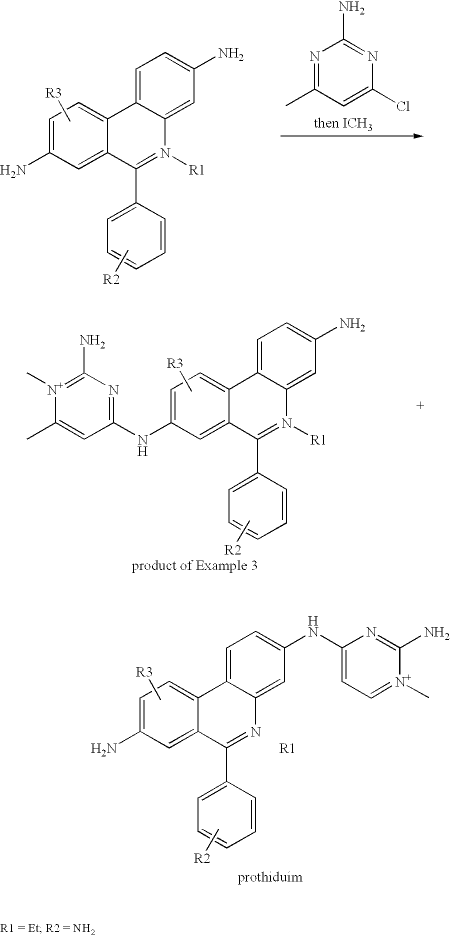

In yet another embodiment, the present invention further relates to therapeutic compositions containing a compound according to the invention, in combination with a support that is pharmaceutically acceptable according to the chosen mode of administration. The pharmaceutical composition can be in solid or liquid form, or in the form of liposomes.

Among solid compositions, mention may be made, for example, of powders, gelatine capsules, and tablets. Among oral forms, solid forms that are protected against the acid medium of the stomach can also be included. The supports used for the solid forms may consist, for example, of inorganic supports such as phosphates or carbonates, or organic supports such as lactose, celluloses, starch, or polymers. The liquid forms may consist, for example, of solutions, suspensions, or dispersions. As a dispersive support, they may contain either water or an organic solvent (ethanol, NMP, or others), mixtures of surfactants and solvents, or mixtures of complexing agents and solvents.

The administered dose of the compounds of the invention may be adjusted by the practitioner by methods known in the art as a function of the route of administration and the condition of the patient.

The compounds of the invention may be administered alone or in a mixture with other anticancer agents. Among the possible combinations, for example, mention may be made of:

alkylating agents, for example, cyclophosphamide, melphalan, ifosfamide, chlorambucil, busulfan, thiotepa, prednimustine, carmustine, lomustine, semustine, streptozotocin, decarbazine, temozolomide, procarbazine, and hexamethylmelamine;

platin derivatives, such as, for example, cisplatin, carboplatin, or oxaliplatin;

antibiotics, such as, for example, bleomycin, mitomycin, or dactinomycin;

antimicrotubular agents, such as, for example, vinblastine, vincristine, vindesine, vinorelbine, or the taxoids (paclitaxel and docetaxel);

anthracyclines, such as, for example, doxorubicin, daunorubicin, idarubicin, epirubicin, mitoxantrone, or losoxantrone;

group I and II topoisomerases, such as, for example, etoposide, teniposide, amsacrine, irinotecan, or topotecan;

fluoropyrimidines, such as, for example, 5-fluorouracil, UFT, IIfloxuridine, or tomudex;

cytidine analogues, such as, for example, 5-azacytidine, cytarabine, or gemcitabine;

adenosine analogues, such as, for example, 6-mercaptopurine, 6-thioguanine, pentostatin, or fludarabine phosphate;

methotrexate and folinic acid; and

enzymes and diverse compounds, such as, for example, L-asparaginase, hydroxyurea, trans-retinoic acid, suramine, dexrazoxane, amifostine, or herceptin, as well as estrogenic and androgenic hormones.

It is also possible to combine radiation treatment with the compounds of the present invention. This treatment can be administered simultaneously, separately, or sequentially. The practitioner would adapt the treatment to the patient to be treated.

The G-quadruplex stabilizing activity can be determined with a method using the formation of a complex with fluorescein, for which the experimental protocol is described below. The compounds of the present invention can thus be used as a fluorescent probe for determining the presence of G-quadruplexes.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Experimental Protocols

Oligonucleotides

All the oligonucleotides, modified or unmodified, were synthesized by Eurogentec SA, Seraing, Belgium. The FAM+DABCYL oligonucleotide carries the catalogue reference OL-0371-0802 and has the sequence: GGGTTAGGGTTAGGGTTAGGG (SEQ ID NO: 1), corresponding to 3.5 repeats of the human telomeric motif (strand rich in G). The fluorescein is attached to the 5′ end and the DABCYL to the 3′ end, via the chemical arms described by Eurogentec. The concentration of the samples was verified by spectrophotometry, recording the absorbance spectrum between 220 nm and 700 nm and using the molar extinction coefficient provided by the supplier.

Buffers

All the experiments were carried out in a 10 mM sodium cacodylate buffer, pH 7.6, containing 0.1 M of lithium chloride (or of sodium chloride). The absence of fluorescent contamination in the buffer was verified beforehand. The fluorescent oligonucleotide was added at the final concentration of 0.2 μM.

Fluorescence measurements

All the measurements of fluorescence were carried out on a Spex Fluorolog DM1B apparatus, using an excitation line width of 1.8 nm and an emission line width of 4.5 nm. The samples were placed in a 0.2×1 cm microquartz cuvette. The temperature of the sample was controlled by an external water bath. The oligonucleotide alone was analysed at 20, 30, 40, 50, 60, 70, and 80° C. The emission spectra were recorded using an excitation wavelength of 470 nm. The excitation spectra were recorded using either 515 nm or 588 nm as the emission wavelength. The spectra were corrected for the response of the instrument using reference curves. Considerable extinction (80-90%) of the fluorescence due to the fluorescein was observed at room temperature. This result is in agreement with an intramolecular folding of the oligonucleotide at 20° C. in the form of a G-quadruplex. This folding induces juxtaposition of the oligonucleotide's 5′ and 3′ ends, which are respectively linked to fluorescein and to DABCYL. When present, this juxtaposition caused the aforementioned phenomenon of extinction of fluorescence, which was used for determining the presence of a G-quadruplex.

Fluorescence Tm

A stock solution of oligonucleotide at the strand concentration of 0.2 μM in a 0.1 M LiCl, 10 mM sodium cacodylate buffer, pH 7.6, was prepared beforehand, heated briefly at 90° C. and slowly cooled to 20° C., and then distributed, in 600 μl aliquots, into the fluorescence cuvettes. 3 μl of water for the control, or 3 μl of the product to be tested (stock at 200 μM, final concentration 1 μM) were then added and mixed. The samples were then left to incubate for at least 1 hour at 20° C. before each measurement. The use of longer incubation times (up to 24 hours) had no influence on the result obtained.

Each experiment allows the measurement of only one sample. The latter was first incubated at an initial temperature of 20° C., brought to 80° C. over 38 minutes, left for 5 minutes at 80° C., and then cooled to 20° C. in 62 minutes. During this time, the fluorescence was measured simultaneously at two emission wavelengths (515 nm and 588 nm) using 470 nm as the excitation wavelength. A measurement was carried out every 30 seconds. The temperature of the water bath was recorded in parallel, and the fluorescence profile as a function of the temperature was obtained from these values. The fluorescence profiles were then normalized between 20° C. and 80° C., and the temperature for which the intensity of emission at 515 nm was the mean of those at high and low temperature was considered Tm. Under these conditions, the Tm of the reference sample without LAW OFFICES ;addition of product was 44° C. in a lithium chloride buffer. This temperature increased to more than 55° C. in a sodium chloride buffer. The addition of a G-quadruplex-stabilizing compound induced an increase in the Tm. This increase was judged to be significant if it was greater than 3° C.

The antitelomerase biological activity was determined using the following experimental protocol:

Preparation of the extract enriched in human telomerase activity

The HL60 leukaemia line was obtained from the ATCC (American Type Culture Collection, Manassas, Va., USA). The cells were cultured in suspension in RPMI 1640 medium containing L-glutamine at 2 mM, 200 U/ml penicillin, 200 μg/ml streptomycin, and 50 μg/ml gentamycin, and supplemented with 10% of heat-inactivated fetal calf serum.

An aliquot of 10⁵ cells was centrifuged at 3000×G, and the supernatant discarded. The cell pellet was resuspended by pipetting several times successively in 200 μl of lysis buffer containing 0.5% CHAPS, 10 mM Tris-HCl, pH 7.5, 1 mM MgCl₂, 1 mM EGTA, 5 mM β-mercaptoethanol, 0.1 mM PMSF, and 10% glycerol, and was conserved in ice for 30 minutes. The lysate was centrifuged at 160,000×G for 20 minutes at 4° C., and 160 μl of the supernatant was recovered. The proteins in the extract were assayed by the Bradford method. The extract was stored at −80° C.

Telomerase activity assay

The inhibition of the telomerase activity was determined using a protocol for extension of the oligonucleotide TS (^{5′}AATCGTTCGAGCAGAGTT^{3′}, SEQ ID NO: 2), in the presence of a cell extract enriched in telomerase activity and compounds of the invention that were added at various concentrations (10,1, 0.1, and 0.01 μg/l). The extension reaction was followed by a PCR amplification of the extension products using the oligonucleotides TS and CXext (^{5′}GTGCCCTTACCCTTACCCTTACCCTAA^{3′}, SEQ ID NO: 3).

The reaction medium was prepared according to the following composition:

| | |
|---|---|
| Tris HCl pH 8.3 | 20 mM |
| MgCl₂ | 1.5 mM |
| Tween 20 | 0.005% (W/V) |
| EGTA | 1 mM |
| DATP | 50 μm |
| DGTP | 50 μm |
| DCTP | 50 μm |
| DTTP | 50 μm |
| Oligonucleotide TS | 2 μg/ml |
| Oligonucleotide CXext | 2 μg/ml |
| Bovine serum albumin | 0.1 mg/ml |
| Taq DNA polymerase | 1 U/ml |
| Alpha-²³P dCTP (3000 Ci/mmol) | 0.5 μl |
| Telomerase extract | 200 ng in a volume of 10 μl |
| Product to be tested or solvent | in a volume of 5 μl |
| Double-distilled water QS | 50 μl |

The oligonucleotides were obtained from Eurogentec (Belgium) and were stored at −20° C. at a stock concentration of 1 mg/ml in distilled water.

The reaction samples were assembled in 0.2 ml PCR tubes and a drop of paraffin oil was deposited on each of the reactions of the experiment before closing the tubes.

The reaction samples were then incubated in a Cetus 4800-type PCR machine according to the following temperature conditions:

15 minutes at 30° C.,

1 minute at 90° C.,

followed by 30 cycles of: 30 seconds at 94° C., 30 seconds at 50° C., and 1 minute 30 seconds at 72° C.,

followed by a final cycle of 2 minutes at 72° C.

For each of the samples, an aliquot of 10 μl was pipetted under the oil layer and mixed with 5 μl of a loading buffer containing:

| TBE | 3X |
|---|---|
| Glycerol | 32% (W/V) |
| Bromophenol blue | 0.03% |
| Xylene cyanol | 0.03% |

The samples were then analyzed by 12% acrylamide gel electrophoresis in a 1X TBE buffer for 1 hour at a voltage of 200 volts, using a Novex electrophoresis system.

The acrylamide gels were then dried on a sheet of Whatmann 3MM paper at 80° C. for 1 hour.

The analysis and the quantification of the reaction products were carried out using an Instantimager machine (Pacard).

For each concentration of a compound of the invention tested, the results were expressed as percentage inhibition of the reaction and calculated based on the untreated enzymatic control and on the enzyme-free sample (blank) according to the following formula:

[(Compound value−blank value)/(Enzymatic control value−blank value)]×100

The compound concentration that induced 50% inhibition of the telomerase reaction (IC50) was determined with the aid of a semi-logarithmic graphic representation of the inhibition values obtained, as a function of each of the compound concentrations tested.

A compound was considered to be active as an antitelomerase agent when the concentration that inhibited 50% of the telomerase reaction was less than 5 μM.

Cytotoxic biological activity on human tumor lines

The A549 human cell line originates from the ATCC (American Type Culture Collection, Manassas, Va., USA). The A549 cells were cultured in a layer in a culture flask in RPMI 1640 medium containing L-glutamine at 2 mM, 200 U/ml penicillin, and 200 pg/ml streptomycin, and supplemented with 10% of heat-inactivated fetal calf serum.

The cells in the exponential growth phase were trypsinized, washed in 1X PBS and seeded into 96-well microplates (Costar) in a proportion of 4×10⁴ cells/ml (0.2 ml/well). The cells were then incubated for 96 hours in the presence of varying concentrations of product to be tested (10,1, 0.1, and 0.01 μg/ml, each point in quadruplicate). 16 hours before the end of the incubation, 0.02% (final concentration) of neutral red was added to each well. At the end of the incubation, the cells were washed with IX PBS and lysed with 1% of sodium laurylsulphate. The cellular incorporation of the dye, which reflects cell growth, was evaluated by spectrophotometry at a wavelength of 540 nm for each sample, using a Dynatech MR5000 reader machine.

For each compound concentration tested, the results were expressed as percentage inhibition of cell growth and calculated based on the untreated control and on the cell-free culture medium (blank) according to the following formula:

[(Compound value−blank value)/(Cell control value−blank value)]×100

The compound concentration that induced 50% inhibition of the telomerase reaction (IC50) was determined with the aid of a semi-logarithmic graphic representation of the inhibition values obtained, as a function of each of the compound concentrations tested.

A compound was considered to be active as a cytotoxic agent when the concentration that inhibited 50% of the growth of the tumor cells tested was less than 20 μM.

The following nonlimiting examples are given in order to illustrate the invention.

### EXAMPLE 1:

Isomethamidium, or 7-(3-amidinophenyl)diazoamino-2-amino-10-ethyl-9-phenylphenanthridinium chloride hydrochloride.

### ELXAMPLE 2:

8-(3-Amidinophenyl)diazo-2,7-diamino-10-ethyl-9-phenylphenanthridinium bromide hydrobromide.

### EXAMPLE 3:

7-Amino-2-(2-amino-1,6-dimethylpyrimidinio-4-yl)-(4-aminophenyl)-10-ethylphenanthridinium bromide.

### EXAMPLE 4:

The G-quadruplexes, antitelomerase, and cytotoxic activities of the various compounds exemplified were determined according to the procedures described above. These activities are reported in Table 1

TABLE 1

| | Fluorescence | TRAP | A459 Cytotox |
|---|---|---|---|
| Example | Δ Tm (° C.) | IC50 (μM) | IC50 (μM) |
| 1 | 9.7 | 0.019 | 3.38 |
| 2 | 7.9 | 0.097 | not determined |
| 3 | 9.6 | 0.047 | 13.4 |

The present invention may be embodied in other specific forms without departing from its spirit or essential characteristics. The described embodiments are to be considered in all respects as illustrative only and not restrictive.

We claim:

1. A method of reducing the activity of telomerase comprising incubating telomerase with at least one compound of formula (I): 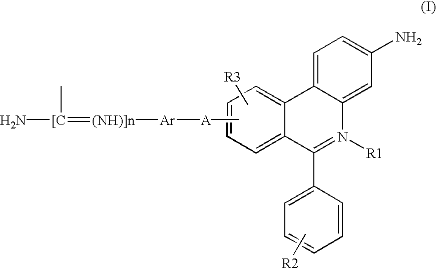
wherein
A represents a substituent with a basic nitrogenous linkage chosen from secondary or tertiary amino, azo, diazo, diazoamino, and hydrazo groups;
Ar represents at least one mono- or polycyclic, carbocyclic or heterocyclic aromatic group containing 4 to 10 carbon atoms, each of said aromatic group being optionally substituted with at least one C1-C4 alkyl group;
n represents 0 or 1;
R1 represents a C1-C4 alkyl group;
R2 represents at least one substituent chosen from hydrogen, C1-C4 alkyl, C1-C4 alkoxy, C1-C4 alkylthio, and an amino group; and
R3 represents at least one substituent chosen from hydrogen, C1-C4 alkyl, C1-C4 alkoxy, C1-C4 alkylthio, and an amino group.

2. A method according to claim 1, wherein at least one of the aromatic heterocycles is a nitrogenous heterocycle comprising at least one nitrogen atom.

3. A method according to claim 2, wherein at least one of the nitrogenous heterocycles is chosen from pyridine, pyrimidine, and pyrazine groups

4. A method according to claim 2, wherein at least one R2 represents a hydrogen or an amino group.

5. A method according to claim 2, wherein R1 represents a methyl group or an ethyl group.

6. A method according to claim 2, wherein n is equal to 1.

7. A method according to claim 1, wherein the compound of formula (I) comprises at least 7 nitrogen atoms.

8. A method according to claim 1, wherein the compound of formula (I) is isomethamidium, or 7-(3-amidinophenyl)diazoamino-2-amino-10-ethyl-9-phenylphenanthridinium chloride hydrochloride.

9. A method according to claim 1, wherein the compound of formula (I) is 8-(3-amidinophenyl)diazo-2,7-diamino-10-ethyl-9-phenylphenanthridinium bromide hydrobromide,

10. A method according to claim 1, wherein the compound of formula (I) is 7-amino-2-(2-amino-1,6-dimethylpyrimidinio-4-yl)-(4-aminophenyl)-10-ethylphenanthridinium bromide.

11. A compound of the following formula: 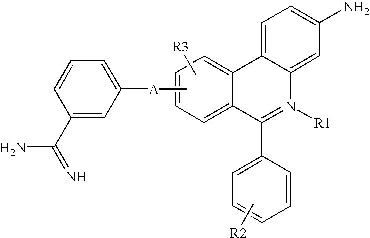
wherein
R1 represents a C1-C2 alkyl group;
R2 represents at least one substituent chosen from hydrogen and an amino group;
R3 represents at least one substituent chosen from hydrogen and an amino group; and
A represents a substituent chosen from an azo, diazo, diazoamino, and hydrazo group.

12. A method for treating cancer comprising administering a composition to patient in need thereof, wherein said composition comprises an effective amount of at least one compound of formula (I) as described in claim 1.

13. The method according to claim 12, wherein the composition further comprises an additional compound for treating cancer.

14. The method according to claim 13, wherein the additional compound for treating cancer is chosen from alkylating agents, platin derivatives, antibiotics, antimicrotubular agents, anthracyclines, group I and II topoisomerases, fluoropyrimidines, cytidine analogues, adenosine analogues, estrogenic and androgenic hormones, L-asparaginase, hydroxyurea, trans-retinoic acid, suramine, irinotecan, topotecan, dexrazoxane, amifostine, and herceptin.

15. The method according to claim 12, further comprising administering radiation itherapy to said patient.

16. The method according to claim 15, wherein the administration of the composition and the administration of radiation therapy occurs simultaneously, separately, or sequentially.

17. A method for inhibiting tumor growth comprising administering a composition to patient in need thereof, wherein said composition comprises an effective amount of at least one compound of formula (I) as described in claim 1.

18. A method for reducing telomerase activity comprising administering a composition to patient in need thereof, wherein said composition comprises an effective amount of at least one compound of formula (I) as described in claim 1.

19. A method for inhibiting cell growth comprising administering a composition to a patient in need thereof, wherein said composition comprises an effective amount of at least one compound of formula (I) as described in claim 1.

20. A method for determining the presence of a G-quadruplex comprising using at least one compound of formula (I) as described in claim 1 as a fluorescent probe.

21. A method for stabilizing a polynucleotide in a G-quadruplex form comprising incubating said polynucleotide with at least one compound of formula (I) as described in claim 1.

22. A method for increasing the Tm of a polynucleotide comprising incubating said polynucleotide with at least one compound of formula (I) as described in claim 1.

23. A method for determining the G-quadruplex-stabilizing activity of a substance comprising:
incubating said substance with a polynucleotide; and
measuring the Tm of said polynucleotide.

24. A method for determining the presence of a polynucleotide in a G-quadruplex form comprising measuring the fluorescence of the polynucleotide-containing solution,
wherein a fluorescent group is attached to one end of said polynucleotide and a non-fluorescent group is attached to the other end of the polynucleotide.

25. A method according to claim 24, wherein the fluorescent group is fluorescein and the non-fluorescent group is DABCYL.


SEQUENCE LISTING (3 sequences)

SEQ ID NO: 1 - DNA, 21 bp, Artificial Sequence
  Description of Artificial Sequence  Synthetic oligonucleotide
gggttagggttagggttaggg

SEQ ID NO: 2 - DNA, 18 bp, Artificial Sequence
  Description of Artificial Sequence  Synthetic oligonucleotide
aatcgttcgagcagagtt

SEQ ID NO: 3 - DNA, 27 bp, Artificial Sequence
  Description of Artificial Sequence  Synthetic oligonucleotide
gtgcccttacccttacccttaccctaa